(19) United States
(12) Patent Application Publication
(10) Pub. No.: US 2019/0284619 A1
(43) Pub. Date: Sep. 19, 2019

(54) IN SITU PROBE INVERSION PROCESS FOR CONTSTRUCTING PROBE ARRAYS

(71) Applicant: Centrillion Technology Holdings Corporation, Grand Cayman (KY)

(72) Inventors: Wei ZHOU, Saratoga, CA (US); Vijay SINGH, Union City, CA (US)

(21) Appl. No.: 16/426,529

(22) Filed: May 30, 2019

Related U.S. Application Data
(63) Continuation of application No. PCT/US2017/064169, filed on Dec. 1, 2017.
(60) Provisional application No. 62/429,658, filed on Dec. 2, 2016.

(51) Int. Cl. C12Q 1/6834; C07H 21/04

(52) U.S. Cl. CPC C12Q 1/6834; C07H 21/04

(57) ABSTRACT

The present disclosure relates to processes for inverting oligonucleotide probes in an in situ synthesized array. These processes can be used to reverse the orientation of probes with respect to the substrate from 3′-bound to 5′-bound. These processes can also be used to reduce or eliminate the presence of truncated probe sequences from an in situ synthesized array.

### CROSS-REFERENCE

This application is a continuation of International Application No. PCT/US2017/064169, filed Dec. 1, 2017, which claims the benefit of U.S. Provisional Patent Application No. 62/429,658, filed on Dec. 2, 2016, all of which are entirely incorporated herein by reference.

### SEQUENCE LISTING

The instant application contains a Sequence Listing which has been filed electronically in ASCII format and is hereby incorporated by reference in its entirety. Said ASCII copy, created on May 30, 2019, is named 38558_731_301 SL.txt and is 4,096 bytes in size.

### BACKGROUND

The synthesis of oligonucleotide probes on in situ synthesized arrays, such as by photolithography, can result in a population of incomplete or truncated probe sequences which accompany the probe sequences synthesized at the full desired or intended length (“full-length” probes). The presence of such truncated probe sequences can have a detrimental effect on array performance, for example, in hybridization reactions to contribute to a poor signal-to-noise ratio. In addition, photolithography permits efficient oligonucleotide synthesis in the 3′ to 5′ direction with the 3′-terminus of the synthesized probe bound to the solid support. Certain enzymatic reactions demanding enzymatic addressing of the free probe terminus, such as polymerase extension reactions or ligation reactions, require a free 3′-hydroxyl which is absent in photolithography-based microarrays.

In contrast, oligonucleotide probes immobilized on bead arrays (e.g., Illumina) and other spotted arrays are commonly attached to their substrates via an amine or other functional groups synthetically attached to the 5′ end of the full-length, previously synthesized and purified probes. In this way, only full-length sequences are immobilized, and truncations or other defects associated with an exposed free 3′ end are reduced or virtually eliminated. But arrays of increased complexity are difficult to be synthesized in this way.

### SUMMARY

It can be desirable to selectively and post-synthetically remove truncated probe sequences from among the probes on in situ synthesized arrays, such as those fabricated with photolithography. The present disclosure can provide processes for accomplishing this selective in-situ removal of truncated sequences, while simultaneously inverting the orientation of the probe sequence such that probe sequences synthesized on the substrate from the 3′ ends to the 5′ ends can be converted to probe sequences attached to the substrate via their 5′ ends with their free 3′-hydroxyls exposed on the probe.

In particular, this disclosure includes probe inversion processes for in situ synthesized arrays which can use universal linkers and commercially available building blocks and reagents. These can include incorporation of a universal cleavable linker (UCL), which upon cleavage releasing a free 3′-OH termini, incorporation of branched linkers (BL) with orthogonally protected, addressable functional groups for oligonucleotide synthesis and post-synthesis circularization, more efficient chemistries for circularization steps, and other improvements. Previous processes attempting probe inversion on in situ synthesized arrays involved a large number of special linkers, building blocks and reagents, which may present difficulties for large scale manufacturing of in situ synthesized arrays. A one-pot procedure for the inversion process, which can result in both circularization of the 5′ end of the oligonucleotide to the substrate and the release of 3′ end of the oligonucleotide from the substrate, can be desirable.

An aspect of the present disclosure provides a method, comprising: (a) providing a substrate; (b) adding 5 DMT-Ts to the substrate; (c) attaching a branched linker to the substrate, wherein the branched linker comprises (i) a first branch comprising an acetylene moiety, and (ii) a second branch comprising a hydroxyl group for further base coupling; (d) attaching a universal cleavable linker to the second branch; (e) synthesizing an oligonucleotide on the universal cleavable linker in 3′ to 5′ orientation using photo labile protecting groups, the oligonucleotide comprising (i) a 3′ end coupled to the second branch via the universal cleavable linker, and (ii) a 5′ end coupled to a bromohexyl linker; (f) in-situ circularizing the oligonucleotide; and (g) in-situ cleaving the cleavable moiety, thereby de-coupling the 3′ end of the oligonucleotide from the second branch. In some embodiments, both in-situ circularizing and in-situ cleaving are conducted with a basic reagent. In some embodiments, the basic reagent is an amine.

In another aspect, disclosed herein is a method of inverting an oligonucleotide on a surface comprising: (a) providing a substrate; (b) attaching a branched linker to the substrate, wherein the branched linker comprises (i) a first branch comprising an acetylene moiety and (ii) a second branch comprising a hydroxyl group for further base coupling; (c) attaching a universal cleavable linker to the second branch; (d) synthesizing an oligonucleotide on the universal cleavable linker in 3′ to 5′ orientation, the oligonucleotide comprising (i) a 3′ end coupled to the second branch via the universal cleavable linker and (ii) a 5′ end coupled to a bromohexyl linker; (e) in-situ circularizing the oligonucleotide to couple the 5′ end of the oligonucleotide to the first branch; and (g) in-situ cleaving the universal cleavable moiety, thereby de-coupling the 3′ end of the oligonucleotide from the second branch. In some embodiments, both in-situ circularizing and in-situ cleaving are conducted with a basic reagent. In some embodiments, the basic reagent is an amine.

In still another aspect, disclosed herein is a method of inverting an oligonucleotide on a surface, comprising: (a) providing a substrate; (b) attaching a branched linker to the substrate, wherein the branched linker comprises (i) a first branch comprising an acetylene moiety, and (ii) a second branch comprising a hydroxyl group; (c) attaching a universal cleavable linker to the second branch via the hydroxyl group on the second branch; (d) synthesizing a first oligonucleotide on the universal cleavable linker in 3′ to 5′ orientation, the first oligonucleotide comprising (i) a 3′ end coupled to the second branch via the universal cleavable linker, and (ii) a 5′ end coupled to a 6-bromohexyl linker; (e) in-situ circularizing the first oligonucleotide by treating with a deprotection reagent, thereby coupling the 5′ end of the first oligonucleotide to the substrate; and (f) in-situ cleaving the universal cleavable linker by the treating with the deprotection reagent, thereby de-coupling the 3′ end of the first oligonucleotide from the second branch.

In some embodiments, the branched linker is coupled to the substrate via a first reagent of

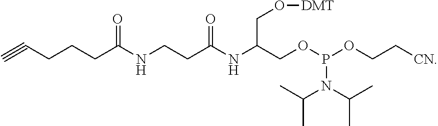

In some embodiments, the universal cleavable linker is coupled to the branched linker via a second reagent of

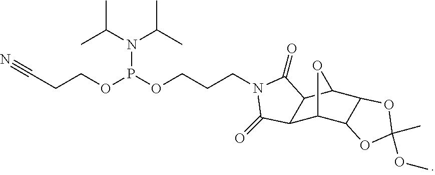 [SMILES: COC1(C)OC2C(O1)C1OC2C2C(=O)N(CCCOP(OCCC#N)N(C(C)C)C(C)C)C(=O)C12]

In some embodiments, the 6-bromohexyl linker is coupled to the first oligonucleotide via a third reagent of

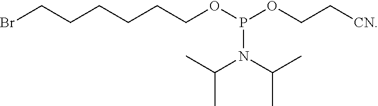 [SMILES: CC(C)N(C(C)C)P(OCCC#N)OCCCCCCBr]

In some embodiments, the deprotection reagent comprises a base. In some embodiments, the base comprises an amine. In some embodiments, the base comprises at least one of: (i) 1,2-diaminoethane, (ii) NH₄OH, and (iii) methyl amine. In some embodiments, the method further comprises in (d) building a second oligonucleotide in 3′ to 5′ orientation on a third branch on the substrate, wherein the second oligonucleotide is shorter than the first oligonucleotide and without another 6-bromohexyl linker coupled to a 5′ end of the second oligonucleotide, and wherein the in-situ cleaving in (f) releases the second oligonucleotide from the substrate.

In still another aspect, disclosed herein is a method of preparing probes on a substrate, comprising: (a) coupling a plurality of first linkers to the substrate, wherein each the first linker is further coupled to a first branch comprising a first cleavable linker coupled to a 3′ end of a first oligonucleotide, and wherein a 5′ end of the first oligonucleotide is coupled to a first reactive group; (b) coupling a plurality of second linkers to the substrate, wherein each the second linker is further coupled to a second branch comprising a second reactive group; and (c) circularizing the first oligonucleotide by reacting the first reactive group with the second reactive group, thereby coupling the 5′ end of the first oligonucleotide to the substrate via the second linker; wherein the circularizing in (c) is conducted using nucleophilic substitution reaction, photo-crosslinking reaction, or radical reaction.

In some embodiments, the circularizing in (c) is conducted using nucleophilic substitution reaction. In some embodiments, the circularizing is conducted using a base. In some embodiments, the base comprises an amine. In some embodiments, the base comprises at least one of: (i) 1,2-diaminoethane, (ii) NH₄OH, and (iii) methyl amine. In some embodiments, the first reactive group comprises at least one of: (i) bromide, (ii) iodide, (iii) mesylate, (iv) tosylate, and (v) triflate. In some embodiments, the second reactive group comprises at least one of: (i) terminal acetylene, (ii) thymidine, (iii) —C(O)NH—, (iv) amine, (v) —OH, (vi) alkene, and (vii) alpha-hydrogen of a carbonyl. In some embodiments, the first reactive group is bromide.

In some embodiments, the method further comprises: after (c), cleaving the first cleavable linker, thereby de-coupling the 3′ end of the first oligonucleotide from the first linker. In some embodiments, both the circularizing and the cleaving are conducted using a base. In some embodiments, the method further comprises: before (c), coupling a plurality of third linkers to the substrate, wherein each the third linker is further coupled to a third branch comprising a second cleavable linker coupled to a 3′ end of a second oligonucleotide, wherein the second oligonucleotide is shorter than the first oligonucleotide, and wherein the cleaving releases the second oligonucleotide from the substrate. In some embodiments, the second oligonucleotide does not comprise the first reactive group at a 5′ end of the second oligonucleotide. In some embodiments, the circularizing in (c) does not couple the 5′ end of the second oligonucleotide to the substrate. In some embodiments, the first linker is the second linker. In some embodiments, the first linker is coupled to the substrate via a first reagent of

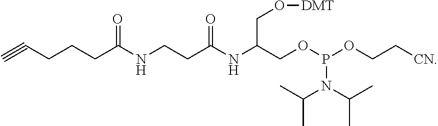

In some embodiments, the first cleavable linker is coupled to the first linker via a second reagent of

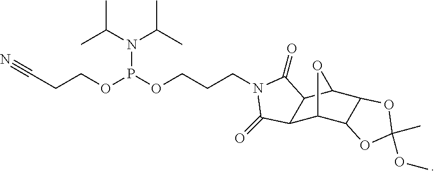 [SMILES: COC1(C)OC2C(O1)C1OC2C2C(=O)N(CCCOP(OCCC#N)N(C(C)C)C(C)C)C(=O)C12]

In some embodiments, the first reactive group is coupled to the first oligonucleotide via a third reagent of

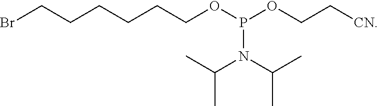 [SMILES: CC(C)N(C(C)C)P(OCCC#N)OCCCCCCBr]

In another aspect, disclosed herein is a method of preparing probes on a substrate, comprising: (a) coupling a plurality of first linkers to the substrate, wherein each the first linker is further coupled to a first branch comprising a first cleavable linker coupled to a 3′ end of a first oligonucleotide, and wherein a 5′ end of the first oligonucleotide is further coupled to a first reactive group; (b) coupling a plurality of second linkers to the substrate, wherein each the second linker is further coupled to a second branch comprising a second reactive group; and (c) circularizing the first oligonucleotide by reacting the first reactive group with the second reactive group, thereby coupling the 5′ end of the first oligonucleotide to the substrate via the second linker; wherein the circularizing in (c) is conducted using a base. In some embodiments, the base comprises an amine. In some embodiments, the base comprises at least one of: (i) 1,2-diaminoethane, (ii) NH₄OH, and (iii) methyl amine.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example process for inverting a probe.

FIG. 2A shows an example phosphoramidite reagent to make a branched linker.

FIG. 2B shows another example phosphoramidite reagent to make a branched linker.

FIG. 2C shows still another example phosphoramidite reagent to make a branched linker.

FIG. 3A shows an example phosphoramidite reagent to make a cleavable linker. FIG. 3B shows another example phosphoramidite reagent to make a cleavable linker. FIG. 3C shows still another example phosphoramidite reagent to make a cleavable linker.

FIG. 4 shows an example phosphoramidite reagent to add a reactive group to an oligonucleotide.

FIG. 5 shows an example diagram for two reactive groups used to invert a probe.

FIG. 6A shows an image of primer extension results of an invertible chip (DNA with all the linkers) without previously undergoing a deprotection step.

FIG. 6B shows an image of primer extension results of an invertible chip (DNA with all the linkers) previously undergoing a deprotection step.

FIG. 6C shows an image of primer extension results of a non-invertible chip (DNA without the linkers) previously undergoing a deprotection step.

### DETAILED DESCRIPTION

The present disclosure provides processes for the inversion of in situ synthesized oligonucleotide probes. The processes disclosed herein can also reduce or eliminate truncated oligonucleotide probes, which do not contain the full synthesized oligonucleotide sequence, while preserving full-length oligonucleotide probes, which do contain the full synthesized oligonucleotide sequence. For example, full-length oligonucleotides can be circularized prior to release of the 3′ ends, while non-full-length oligonucleotides remain un-circularized and therefore are removed from the surface upon release of the 3′ ends.

The terminology used herein is for the purpose of describing particular cases only and is not intended to be limiting. As used herein, the singular forms “a”, “an” and “the” can be intended to include the plural forms as well, unless the context clearly indicates otherwise. Furthermore, to the extent that the terms “including”, “includes”, “having”, “has”, “with”, or variants thereof can be used in either the detailed description and/or the claims, such terms can be intended to be inclusive in a manner similar to the term “comprising”.

The term “about” or “approximately” can mean within an acceptable error range for the particular value as determined by one of ordinary skill in the art, which will depend in part on how the value is measured or determined, i.e., the limitations of the measurement system. For example, “about” can mean about plus or minus 10%, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, or within 2-fold, of a value. Where particular values may be described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “substantially” as used herein can refer to a value approaching 100% of a given value. For example, an active agent that is “substantially localized” in an organ can indicate that about 90% by weight of an active agent, salt, or metabolite can be present in an organ relative to a total amount of an active agent, salt, or metabolite. In some cases, the term can refer to an amount that can be at least about 90%, 91%, 92%, 93%, 94%, 95%, 96%, 97%, 98%, 99%, 99.9%, or 99.99% of a total amount. In some cases, the term can refer to an amount that can be about 100% of a total amount.

The term “oligonucleotide” as used herein can refer to a nucleotide chain. In some cases, an oligonucleotide is less than 200 residues long, e.g., between 15 and 100 nucleotides long. The oligonucleotide can comprise at least or about 1, 2, 3, 4, 5, 6, 7, 8, 9, 10, 15, 20, 25, 30, 35, 40, 45, or 50 bases. The oligonucleotides can be from about 3 to about 5 bases, from about 1 to about 50 bases, from about 8 to about 12 bases, from about 15 to about 25 bases, from about 25 to about 35 bases, from about 35 to about 45 bases, or from about 45 to about 55 bases. The oligonucleotide (also referred to as “oligo”) can be any type of oligonucleotide (e.g., a primer). Oligonucleotides can comprise natural nucleotides, non-natural nucleotides, or combinations thereof.

The term “circularize” or “circularization” as used herein can refer to the structure of an oligonucleotide with both of its ends attached to the substrate or support via covalent bonds.

The term “phosphotriester chemistry” as used herein for circularization can refer to

Genetic information can be utilized in a myriad of ways with the advent of rapid genome sequencing and large genome databases. One of such applications is oligonucleotide arrays. The general structure of an oligonucleotide array, or commonly referred to as a DNA microarray or DNA array or a DNA chip, is an array of spots or addressable locations on a surface. Each spot can contain a layer of relatively short strands of DNA called “probe” or “capture probe” (e.g., Schena, ed., “DNA Microarrays A Practical Approach,” Oxford University Press; Marshall et al. (1998) Nat. Biotechnol. 16:27-31; each incorporated herein by reference). There are at least two technologies for generating arrays. One is based on photolithography (e.g. Affymetrix) while the other is based on robot-controlled ink jet (spotbot) technology (e.g., Arrayit.com). Other methods for generating microarrays may be used herein.

Generally, an oligonucleotide (probe or capture probe) placed within a given spot in the array can be selected to bind at least a portion of a nucleic acid or complimentary nucleic acid of a target nucleic acid. An aqueous sample can be placed in contact with the array under the appropriate hybridization conditions. The array then can be washed thoroughly to remove all non-specific adsorbed species. In order to determine whether or not the target sequence has been captured, the array can be “developed” by adding, for example, a fluorescently labeled oligonucleotide sequence that is complimentary to an unoccupied portion of the target sequence. The microarray then can be “read” using a microarray reader or scanner, which outputs an image of the array. Spots that exhibit strong fluorescence can be positive for that particular target sequence.

A probe can comprise biological materials deposited so as to create spotted arrays. A probe can comprise materials synthesized, deposited, or positioned to form arrays according to other technologies. Thus, microarrays formed in accordance with any of these technologies may be referred to generally and collectively hereafter for convenience as “probe arrays.” The term “probe” as used herein is not limited to probes immobilized in array format. Rather, the functions and methods described herein can also be employed with respect to other parallel assay devices. For example, these functions and methods may be applied when probes are immobilized on or in beads, optical fibers, or other substrates or media.

In methods and systems of the present disclosure, probes can be attached to a solid substrate. Probes can be bound to a substrate directly or via a linker. Linkers can comprise, for example, amino acids, polypeptides, nucleotides, oligonucleotides, or other organic molecules that do not interfere with the functions of probes.

The solid substrate can be biological, non-biological, organic, inorganic, or a combination of any of these. The substrate can exist as one or more particles, strands, precipitates, gels, sheets, tubing, spheres, containers, capillaries, pads, slices, films, plates, slides, or semiconductor integrated chips, for example. The solid substrate can be flat or can take on alternative surface configurations. For example, the solid substrate can contain raised or depressed regions on which synthesis or deposition takes place. In some examples, the solid substrate can be chosen to provide appropriate light-absorbing characteristics. For example, the substrate can be a polymerized Langmuir Blodgett film, functionalized glass (e.g., controlled pore glass), silica, titanium oxide, aluminum oxide, indium tin oxide (ITO), Si, Ge, GaAs, GaP, SiO₂, SiN₄, modified silicon, the top dielectric layer of a semiconductor integrated circuit (IC) chip, or any one of a variety of gels or polymers such as (poly)tetrafluoroethylene, (poly)vinylidenedifluoride, polystyrene, polycarbonate, polydimethylsiloxane (PDMS), polymethylmethacrylate (PMMA), polycyclicolefins, or combinations thereof.

Solid substrates can comprise polymer coatings or gels, such as a polyacrylamide gel or a PDMS gel. Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, betaine acrylamide monomers, and combinations thereof.

DNA microarrays can be fabricated using spatially-directed in situ synthesis or immobilization of pre-synthesized oligonucleotides. In both cases, synthesis of the oligonucleotides typically can proceed with the addition of monomers in the 3′-to-5′ direction, using standard 3′-phosphoramidite reagents and solid-phase synthesis protocols (e.g., M. Egli, et al., ed. “Current Protocols in Nucleic Acid Chemistry,” John Wiley & Sons). The main impurities are truncated, partial-length sequences resulting from incomplete monomer coupling and, to a lesser extent, depurination reactions.

On the one hand, fabricating arrays of pre-synthesized oligonucleotide probes typically can involve covalent attachment of the oligonucleotides to a substrate through the 5′-terminus, via a reactive modifier which can be added to the end when the oligonucleotides are synthesized on high-throughput synthesizers (see S. J. Beaucage, et al., Curr. Med. Chem. 2001, 8, 1213-44). This ensures that the probes which are attached to the support can be primarily full-length sequences, since truncated sequences can be capped and rendered non-reactive during synthesis (Brown T and Brown T, Jr. (2005-2015) Solid-phase oligonucleotide synthesis. [Online] Southampton, UK, ATDBio. <http://www.atdbio.com/content/17/Solid-phase-oligonucleotide-synthesis>[Accessed Aug. 9, 2016]).

An advantage of the present disclosure can be that the 3′-hydroxyl group of the oligonucleotide probe is “distal” to the substrate, and can be freely available for enzymatic reactions, such as template-directed polymerase-catalyzed chain extension (or primer extension) and ligation; and this character can be exploited to carry out very sensitive and specific assays for detecting and quantitating genetic polymorphisms (K. Lindroos, et al., Nucleic Acids Res. 2001, 29, e69; Gunderson K L, et al., Nature Genetics 2005, 37, 549-54).

On the other hand, DNA microarrays can also be fabricated using in situ synthesis of sequences directly on the support. In this case, sequences can be “printed” in a highly parallel fashion by spatially-directing the synthesis using inkjet (T. R. Hughes, et al., Nature Biotechnol 2001,19,342-7; C. Lausted, et al., Genome Biol 2004, 5, R58), photolithographic technologies (A. C. Pease, et al., Proc Natl Acad Sci USA 1994, 91, 5022-6; G. McGall, et al., Proc Natl Acad Sci USA 1996; 93:13555-60; S. Singh-Gasson, et al., Nature Biotechnol 1999, 17, 974-8;), or electrochemical techniques (PLoS ONE 2006, 1, e34; B. Y. Chow, et al., Proc Natl Acad Sci USA 2009, 106, 15219-24). Here too, synthesis proceeds in the 3′ to 5′ direction (solid-phase oligonucleotide synthesis in the 5′-to-3′ direction, while feasible, can be much less efficient and economical, providing lower yields and product purity). However, the resulting probes can be attached to the substrate at the 3′-terminus, and any truncated sequence impurities which arise during the synthesis remain on the support, which may be a particular issue in the case of photolithographic synthesis (J. Forman, et al., Molecular Modeling of Nucleic Acids, Chapter 13, p. 221, American Chemical Society (1998) and (1 McGall, et al., J. Am. Chem. Soc. 119:5081-5090 (1997)). As a result, polymerase-based extension assays normally are not feasible using arrays made this way and with this direction (5′ to 3′).

Despite the above limitation, photolithographic synthesis can be a highly attractive means of fabricating very high-density DNA arrays, as it can be capable of exceeding 10 million arrayed sequences per cm² (A. R. Pawloski, et al., J Vac Sci Technol B 2007, 25, 2537-46), and can be highly scalable in a manufacturing setting. Thus, it can be desirable to develop an effective method of inverting the sequences on such probe arrays.

The plurality of probes can be located in one or more addressable regions (spots, locations, etc.) on a solid substrate, herein generally referred to as “pixels.” In some cases, a solid substrate comprises at least about 2, 3, 4, 5, 6, or 7-10, 10-50, 50-100, 100-500, 500-1,000, 1,000-5,000, 5,000-10,000, 10,000-50,000, 50,000-100,000, 100,000-500,000, 500,000-1,000,000 or over 1,000,000 pixels with probes. In some cases, a solid substrate comprises at most about 2, 3, 4, 5, 6, or 7-10, 10-50, 50-100, 100-500, 500-1,000, 1,000-5,000, 5,000-10,000, 10,000-50,000, 50,000-100,000, 100,000-500,000, 500,000-1,000,000 or over 1,000,000 pixels with probes. In some cases, a solid substrate comprises about 2, 3, 4, 5, 6, or 7-10, 10-50, 50-100, 100-500, 500-1,000, 1,000-5,000, 5,000-10,000, 10,000-50,000, 50,000-100,000, 100,000-500,000, 500,000-1,000,000 or over 1,000,000 pixels with probes.

In some cases it can be useful to have pixels which do not contain probes. Such pixels can act as control spots in order to increase the quality of the measurement, for example, by using binding to the spot to estimate and correct for non-specific binding. In some cases, the density of the probes can be controlled to either facilitate the attachment of the probes or enhance the ensuing detection by the probes.

In some examples, it can be useful to have redundant pixels which have identical probe sequences to another pixel but physically may not be adjacent or in proximity to the other pixel. The data acquired by such probe arrays may be less susceptible to fabrication non-idealities and measurement errors.

In some cases, labels can be attached to the probes within the pixels, in addition to the labels can be incorporated into the targets. In such systems, captured targets can result in two labels coming into intimate proximity with each other in the pixel. As discussed before, interactions between specific labels can create unique detectable signals. For example, when the labels on the target and probe, respectively, are fluorescent donor and acceptor moieties that can participate in a fluorescent resonance energy transfer (FRET) phenomenon, FRET signal enhancement or signal quenching can be detected.

### Synthesis of Inverted Oligonucleotides

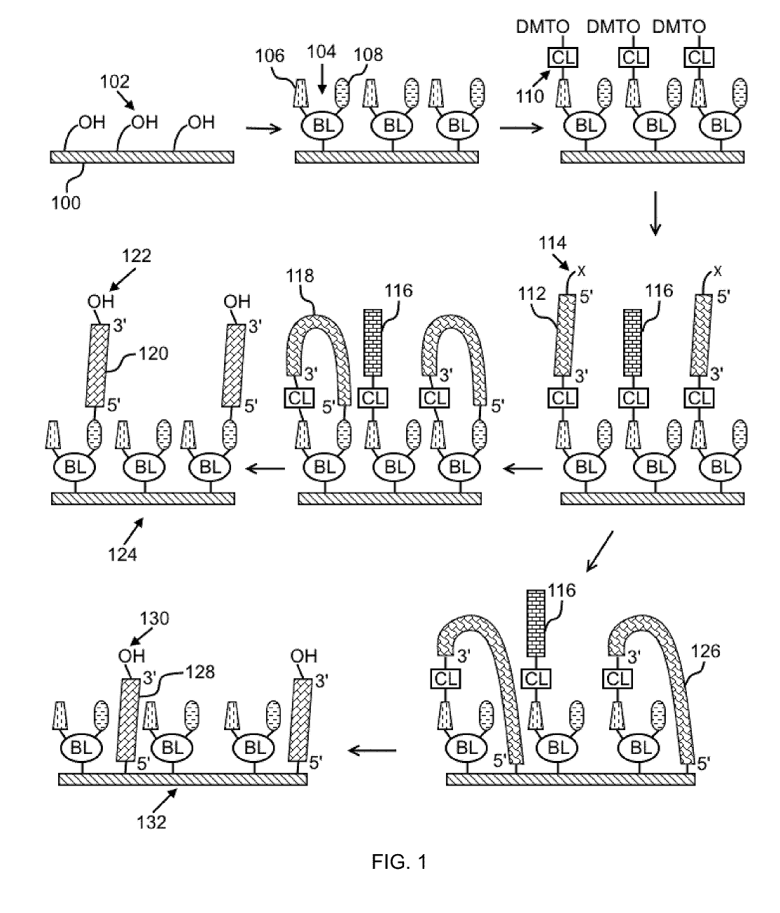

An example process for in situ inversion of a probe is shown in FIG. 1. The synthesis substrate 100, for example, a chip surface, can be cleaned and treated to introduce surface hydroxyl groups 102, which are available for further chemical reactions. In some embodiments, these hydroxyl groups 102 can be part of hydroxyalkyl groups on the synthesis substrate or support. For example, a surface may be modified with reagents, e.g., hydroxyalkyltrialkoxysilane, to provide hydroxyalkyl groups. Alternatively, a polymer thin film can be applied to the surface of the synthesis substrate, wherein the polymer contains free hydroxyl groups. Furthermore, a synthesis substrate 100 that can be compatible with DNA synthesis reagent and processing conditions and that contains hydroxyalkyl groups can be used as well.

In some embodiments, the surface treatment of synthesis substrate 100 can comprise binding oligothymidine groups covalently to the synthesis substrate 100. In some embodiments, the oligothymidine group thus attached to the surface can comprise 2, 3, 4, 5, 6, 7, 8, 9, 10, 11, 12, 13, 14, 15, or more thymidine nucleotides. In some embodiments, the oligothymidine group can comprise 5 thymidine nucleotides. In some embodiments, the free 5′ hydroxyl groups of the oligothymidine group can react with branched linker phosphoramidite and can be covalently attached thereto.

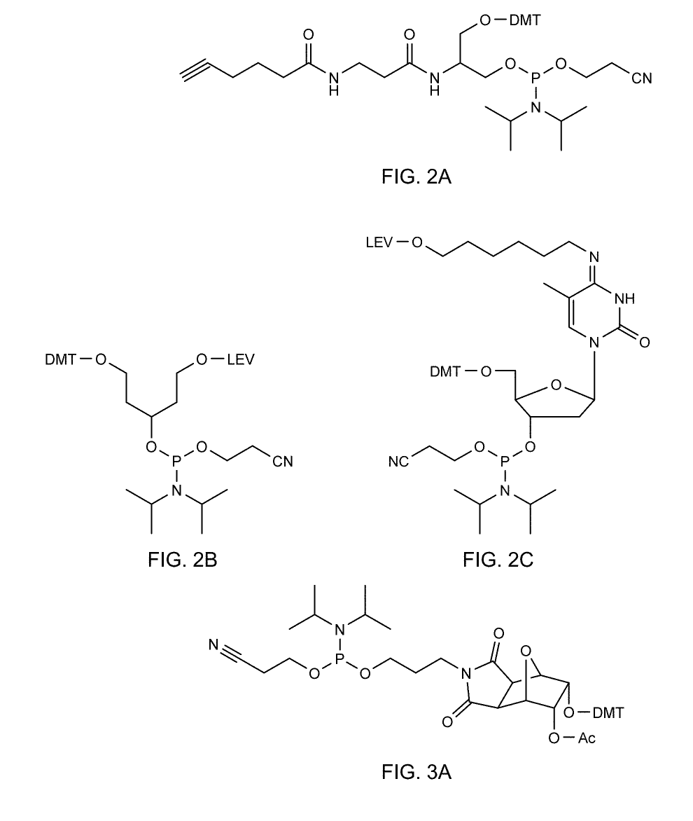

After the surface cleaning and treatment, including adding oligothymidine groups to the surface, a branched linker (BL) phosphoramidite can be immobilized to the surface via the surface hydroxyl groups 102 to form branched linkers 104, which comprise a first branch 106 and a second branch 108, as shown in FIG. 1. The oligothymidine group can comprise five thymidines in-between the surface hydroxyl group 102 and the branched linker 104. Branches 106 and 108 on the branch linker 104 can be chemically distinguishable. Choices for the reagent branched linker phosphoramidite can include, but are not limited to, molecules shown in FIG. 2A, FIG. 2B, and FIG. 2C. As used herein, the term “branched linker”, or BL, can refer to any of the following: a branched linker phosphoramidite reagent, a surfaced-bound branched linker before further attachment of any chemical moiety to the branched linker, and a surfaced-bound branched linker after the addition of any chemical moiety to the branched linker.

Branched linker phosphoramidite can react with active groups on the oligothymidine, for example, free hydroxyl groups at the 5′ end of the oligothymidine, using standard DNA synthesis protocols with some modifications, including, for example, adding the branched linker phosphoramidite reagent to the DNA synthesis substrate, increasing the coupling time (e.g., 3 minutes), etc.

In some embodiments, the first branch 106 comprises a free hydroxyl group or a protected hydroxyl group that can be selectively deprotected in the presence of other protected hydroxyl groups. In some embodiments, the first branch 106 comprises a DMT-protected hydroxyl group (to attach an oligonucleotide later).

In some embodiments, the second branch 108 can comprise a terminal alkyne group. 4,4′-Dimethoxytrityl (DMT) is another hydroxyl protecting group that can be removed under acidic conditions. 2-Cyanoethyl group is a phosphite/phosphate protecting group and can be removed under basic conditions. Levulinoyl ester (LEV) is a protecting group for hydroxyl groups and can be specifically removed using a reagent containing hydrazine hydrate, acetic acid and pyridine (e.g., with 0.5 M hydrazine hydrate in 1:1 mixture of pyridine/acetic acid). The molecules shown in FIGS. 2A, 2B, and 2C are commercially available from Glen Research (Sterling, Va. 20164). It should be noted that the phosphite ester formed after a phosphoramidite reagent reacted with a hydroxyl group can be oxidized to afford the corresponding phosphate ester using iodine reagent.

In some embodiments, a DMT protected primary hydroxyl group can be on the first branch 106 while a LEV protected primary hydroxyl group can be on the second branch 108, thereby providing orthogonally protected hydroxyl groups on the first and second branches. Although this orthogonally protected hydroxyl groups can directly go to the oligonucleotide synthesis step, in cases when further modification of the second branch 108 is needed, the following steps can be taken. First, treatment with hydrazine solution selectively can remove LEV group on the second branch 108, without affecting the DMT group on the first branch 106. Second, the freed hydroxyl group on the second branch 108 can react with compounds of Formula I, shown below, to introduce a masked reactive group (MRG) on the second branch 108.

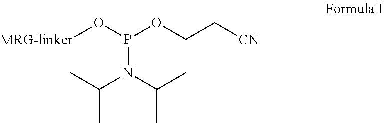

wherein linker is C₂-C₁₀ alkylene, C₂-C₁₀ alkoxylene, or —(CH₂)ₚ—(CH₂CH₂O)ₙ—(CH₂)ₘ—, n is an integer from 1 to 10, m is an integer from 0 to 4, p is an integer from 0 to 4;
MRG is R₁—O—, acetylenenyl, a carbon-carbon double bond, R₂NH—, a group comprising a carbonyl with alpha-hydrogen(s) or a thymidine derivative that do not have available 3′-OH for extension;
R₁ is an orthogonal protecting group for hydroxyl groups with respect to DMT protecting group; and
R₂ is an orthogonal protecting group for amino groups with respect to DMT protecting group.

In some embodiments, R₁ can be acetates, or esters, including, for example, levulinoyl ester (LEV). In some embodiments, R₁ can be removed under basic conditions, including, for example, treatment with ammonium hydroxide, ethylenediamine, or hydrazine. In some embodiments, R₂ can be 9-fluorenylmethyloxycarbonyl (Fmoc) group, or trimethylsilylethoxymethyl (SEM) group. In some embodiments, R₁ can be removed under basic conditions, including, for example, treatment with ammonium hydroxide, ethylenediamine, methylamine, cyclohexylamine, ethanolamine, piperazine, or piperidine.

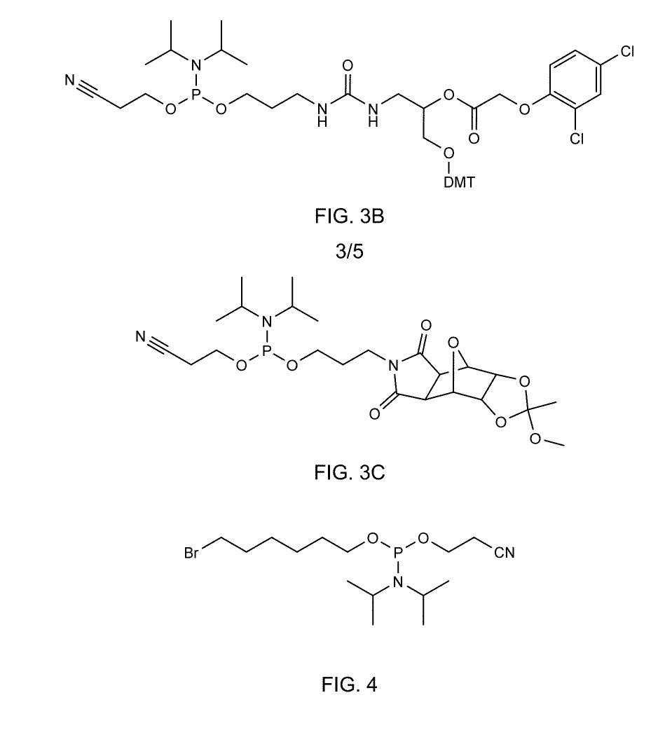

After the introduction of the branched linker 104 (and after the modification of the second branch 108 as shown above, if needed), the first branch 106 can react with a cleavable linker (CL) phosphoramidite through a reactive group on the first branch 106, including, for example, a hydroxyl group. The cleavable linker (CL) phosphoramidite, including, for example, as a universal cleavable linker (UCL) phosphoramidite, can react with the branched linker 104 on its first branch 106 to add the cleavable linker 110, as shown in FIG. 1. Choices for the cleavable linker phosphoramidite can include, but are not limited to, molecules shown in FIG. 3A, FIG. 3B, and FIG. 3C (also known as UCL). As used herein, the term cleavable linker, or CL (including UCL), can refer to any of the following: a cleavable linker phosphoramidite reagent, a surfaced-bound cleavable linker before the addition of nucleotides, and a surfaced-bound branched linker after the addition of nucleotides. Cleavable linker phosphoramidite can react with the first branch 106 of the branched linker 104 using standard DNA synthesis protocols with some modifications, including, for example, adding the cleavable linker reagent to the DNA synthesis substrate, increasing the coupling time (e.g., 3 minutes), etc. In some embodiments, the cleavable linker phosphoramidite can react with free hydroxyl groups on the first branch 106 of the branched linker 104. In some embodiments, the cleavable linker can comprise a hydroxyl group protected by DMT. In some embodiments, the cleavable linker can comprise a primary hydroxyl group protected by DMT.

In some embodiments, the primary hydroxyl group with DMT on the cleavable linker 110 can be deprotected after the installation of the cleavable linker 110 onto the branched linker 104. Standard oligonucleotide probe synthesis can then be conducted on the cleavable linker 110 via its free hydroxyl group to synthesize probe sequences. The synthesized probe sequences can comprise full-length probe sequences 112 and truncated probe sequences 116. Full-length probe sequences 112 can comprise a linker group on the 5′ end with a leaving group X, as shown in FIG. 1, for example, a bromide group or an iodide group or other leaving groups at that end. For example, the last coupling of a DNA synthesis protocol can be performed with 5′-bromohexyl phosphoramidite (6-bromo-hex-1-yl-(2-cyanoethyl)-(N,N-diisopropyl)-phosphoramidite, shown in FIG. 4) to provide a bromide group at the 5′ end of the synthesized oligonucleotides. In some cases, full-length probe sequences 112 can comprise an —OH group on the 5′ end, and the —OH group can react with reagents to add a linker with desired leaving group X (such as Br, I, tosylate, mesylate, and triflate) on its end. In contrast, the truncated probe sequence 116 does not comprise a leaving group attached to its 5′ end. Such a difference in chemical structure/activity can be utilized in the ensuing probe inversion and cleavage of truncated probes.

In some embodiments, the leaving group X on the full-length probe sequence 112 can be the handle through which the probe inversion process, including the circularization step, can be accomplished. In some embodiments, a nucleophile can be provided on the surface of substrate 100 or on the second branch 108 of the branched linker 104. The nucleophile, including, for example, a hydroxyl group, an amino group, or a methylene in a 1,3-dicarbonyl moiety, can displace the leaving group on the 5′ end of the oligonucleotide 112 under basic conditions, thereby circularizing the oligonucleotide to the surface of substrate 100 or the second branch 108. In some embodiments, the leaving group X may lead to a radical at the 5′ end of the full-length probe sequence 112. The radical can form a covalent bond with an unsaturated functional group on the surface of substrate 100 or the second branch 108 on the branched linker 104. In contrast, the truncated probe sequence 116 cannot form covalent bond with either the surface of substrate 100 or the second branch 108 on the branched linker 104. Consequently, circularization can only be conducted with full-length probe sequences, not with the truncated probe sequences.

Back to FIG. 1, circularization can produce a circularized probe sequence 118 between the cleavable linker 110 and the second branch 108, followed by cleavage of the cleavable linker 110 under basic conditions to release the 3′-OH termini of all probe sequences. Accordingly a full-length probe sequence 120 with a 5′ to 3′ orientation can be produced on the surface of the substrate. A free 3′ hydroxyl group 122 can be on the full-length probe sequence 122 while all truncated probe sequences 116 can be removed after washing. After the global deprotection of cyanoethyl groups from the phosphotriesters of the probe sequences 120 and the remaining linkers, a chip 124 with DNA library composed of full-length probe sequences 120 with 3′ hydroxyl group 122 can be obtained.

Alternatively, as shown in FIG. 1, circularization can also produce a circularized probe sequence 126 between the cleavable linker 110 and the surface of substrate 100, followed by cleavage of the cleavable linker 110 under basic conditions to release the 3′-OH termini of all probe sequences. Accordingly a full-length probe sequence 128 with a 5′ to 3′ orientation can be produced on the surface of the substrate. A free 3′ hydroxyl group 130 can be on the full-length probe sequence 128 while all truncated probe sequences 116 can be removed after washing. After the global deprotection of cyanoethyl groups from the phosphotriesters of the probe sequences 128 and the remaining linkers, a chip 132 with DNA library composed of full-length probe sequences 128 with 3′ hydroxyl group 130 can be obtained.

In some embodiment, the cleavable linker can be cleaved, for example, by reaction with NH₄OH, potassium carbonate, methyl amine, 1,2-diaminoethane (also known as ethylenediamine, EDA), potassium hydroxide in methanol, or AMA (a mixture of NH₄OH and methyl amine in water, e.g., a 1:1 mixture of 40% methylamine in water and NH₄OH). Cleavage of the cleavable linker can release the 3′-OH terminus of all probe sequences, thereby releasing truncated probe sequences 116 from the substrate as well as inverting circularized full-length probe sequences 112 to give full-length probe sequences 120 and 128, respectively, as shown in FIG. 1. It should be noted that when the circularization is conducted under basic conditions, in some embodiments, cleavage of the cleavable linker can happen under the same basic conditions as the circularization. However, if the circularization reaction is not conducted under basic conditions or if the basic condition used does not completely cleave the cleavable linker or to completely deprotect the cyanoethyl protecting group, a separate cleavage/deprotection reaction under basic condition may be needed. In some embodiments, the circularization reaction and the cleavage reaction of the cleavable linker can occur under the same base treatment conditions. In some embodiments, the cleavage reaction of the cleavable linker can occur under different base treatment conditions from those for the circularization reaction. In some embodiments, the deprotection reaction and the cleavage reaction of the cleavable linker can occur under the same base treatment conditions. In some embodiments, the cleavage reaction of the cleavable linker can occur under different base treatment conditions from those for the deprotection reaction.

In some embodiments, the cleavable linker can undergo cleavage under basic conditions, as shown in FIG. 1, to cleave both full-length and truncated probe sequences from their 3′ end. Because of prior circularization provided a covalent bond between the 5′ end of the full-length probe sequences, these probes can be inverted on the surface of the substrate to a 5′ to 3′ orientation. Meanwhile, the truncated probe sequences can be deleted from the solid surface and their only attachment to the substrate can be severed, thereby removing the truncated probe sequences from the substrate after washing. Consequently, in some embodiments, probe sequences left on the substrate can comprise mostly full-length probe sequences with 5′ to 3′ orientation. In some embodiment, the probe inversion step can increase the percentage of full-length probe sequences among all probe sequences when compared with the probes before the probe inversion step.

There may be several advantages of in situ probe inversion disclosed in the present disclosure. It may avoid the use of toxic reagents in certain chemical reactions since the inversion reaction of the present disclosure can occur under the same base treatment in the circularization step. In addition, because the cleavage reaction and the probe inversion reaction can happen under the same base treatment, an extra cleavage step may be eliminated from the probe sequence synthesis to simplify the procedure. As a result, probe inversion can directly be done just after DNA array synthesis such that this in-situ probe inversion can become a straightforward process and can lead to increased signal intensity for inverted probes due to less chance to lose full-length probes. Furthermore, avoiding a separate cleavage step after DNA array synthesis may save time and reduce cost when applied at a larger scale. Removing a synthetic step may decrease operational mistakes which may occur during DNA array preparations. Finally, as there are no post-synthetic steps involved other than the cleavage step, the in-situ probe inversion processes may become easier to operate when compared with other probe inversion processes.

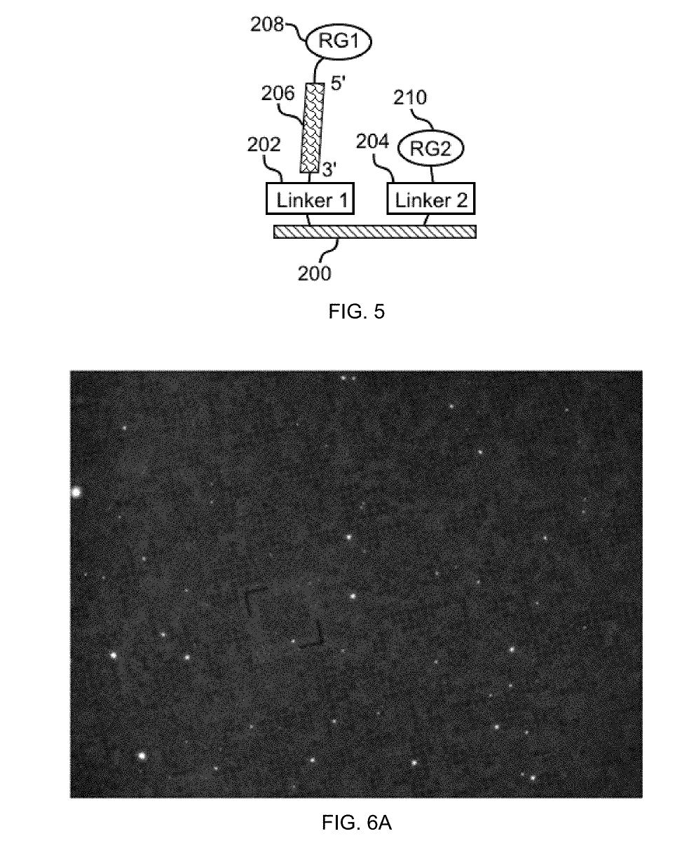

Turning now to FIG. 5, an example diagram of the circularization reaction of a full-length probe sequence is shown. The synthesis substrate 200, for example, a chip surface, can be covalently attached to a first linker 202 and a second linker 204. Either or both linkers 202 and 204 can comprise an oligothymidine portion linked to the synthesis substrate 200. The first linker 202 can be covalently attached to the 3′ end of a full-length probe sequence 206. The 5′ end of the full-length probe sequence 206 can be covalently attached to a first reactive group 208. A second reactive group 210 can be covalently attached to the linker 202. Furthermore, the first reactive group 208 and the second reactive group 210 can form a covalent bond between them, thereby circularizing the full-length probe sequence 206 with the substrate 200.

In some embodiments, both the first linker 202 and the second linker 204 can be the same linker. In some embodiments, the first linker 202 and the second linker 204 can different linkers. In some embodiments, the first linker 202 can be a linker while the second linker 202 is void or a part of the synthesis substrate 200, i.e., the second reactive group 210 is coupled directly to the surface of the synthesis substrate 200 without any linker in-between them.

Example circularization reactions between the first reactive group 208 and the second reactive group 210 can be found in Scheme 1 below. In Scheme 1, the first reactive group is represented by RG1-CH₂X, or RG1 and its attached functional group, wherein X is a leaving group, such as, for example, Br, I, tosylate, mesylate, or triflate, R′ can be C₁-C₆ alkyl, C₁-C₆ alkoxy, halide, CN, or other functional groups. The second reactive group is represented by RG2 and its attached functional groups as shown in Scheme 1.

Reactions 1)-6) can be nucleophilic substitution reactions between the leaving group X of the first reactive group and the respective nucleophile of the second reactive group in each reaction under basic conditions. R₁₀ in reaction 5) can be C₁-C₆ alkyl or Cᵢ-C₆ alkoxy, or R₁₀ can be a linker to the substrate, wherein the linker is C₁-C₂₀ alkylene or C₁-C₂₀ alkoxylene, with or without substitutions such as halide, CN, C₁-C₄ alkoxy groups. The base reagents used in the nucleophilic substitution reactions can comprise at least one of Na₂CO₃, K₂CO₃, ammonium hydroxide, ethylenediamine, trimethylamine, diisopropylethyl amine, 1,8-diazabicyclo[5.4.0]undec-7-ene, NaOEt, NaOCH₃, and NaH. The thymine molecule in reaction 6) can be from the oligothymidine groups covalently to the synthesis substrate 200 as discussed above.

Reactions 7) and 8) can be radical addition reactions. UV light radiation, heating, and/or addition of radical initiators such as, for example, benzoyl peroxide and 2,2′-azobis(2-methylpropionitrile), can generate radicals on the first reactive group when the leaving group X is Br or I. The radicals thus generated can add to the unsaturated double or triple bonds on the second reactive group for chain propagation and the formation of covalent bonds.

Reaction 9) can be photo-crosslinking of part of the probe sequence or a reactive group attached to the 5′-end of the probe sequence with a thymidine residue from the oligothymidine groups covalently to the synthesis substrate 200 as discussed above. It should be noted that the photo-crosslink is between one unsaturated carbon-carbon bond with another unsaturated carbon-carbon bond, wherein the unsaturated carbon-carbon bond can be an alkene or alkyne with or without further substitutions at each carbon. For example, one unsaturated carbon-carbon bond can be from one of the nucleosides within the probe sequence. The term “photo-crosslinking” as used herein can refer to the formation of a covalent bond between two molecules or between two different parts of one molecule. For example, photo-crosslinking reactive group, such as, for example, p-azidophenacyl and 3-cyanovinylcarbazole, can be covalently linked to other unsaturated reactive groups, including a thymidine.

The reaction types shown in Scheme 1 are examples and are not exclusive. Other types of circularization reactions can still be possible.

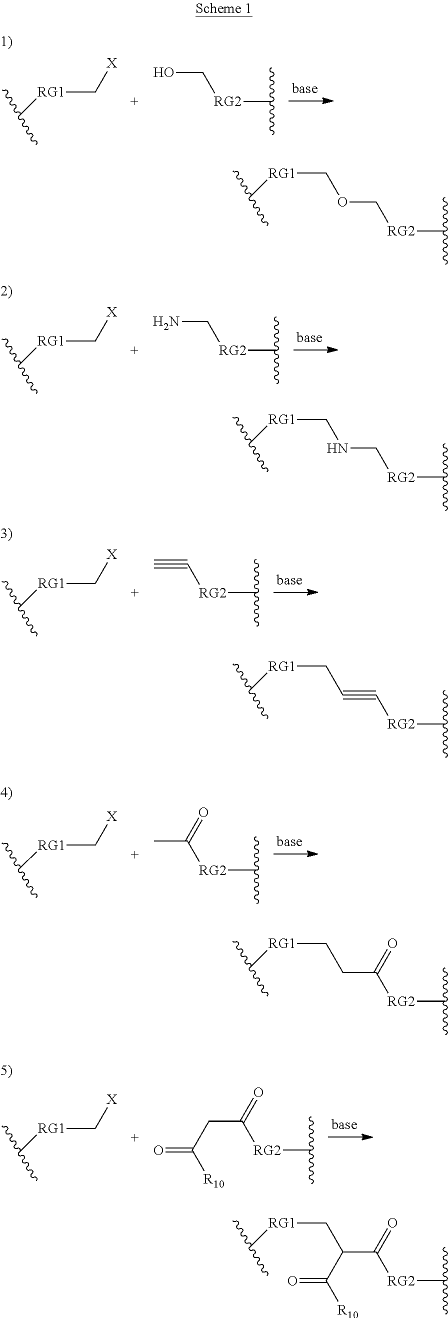

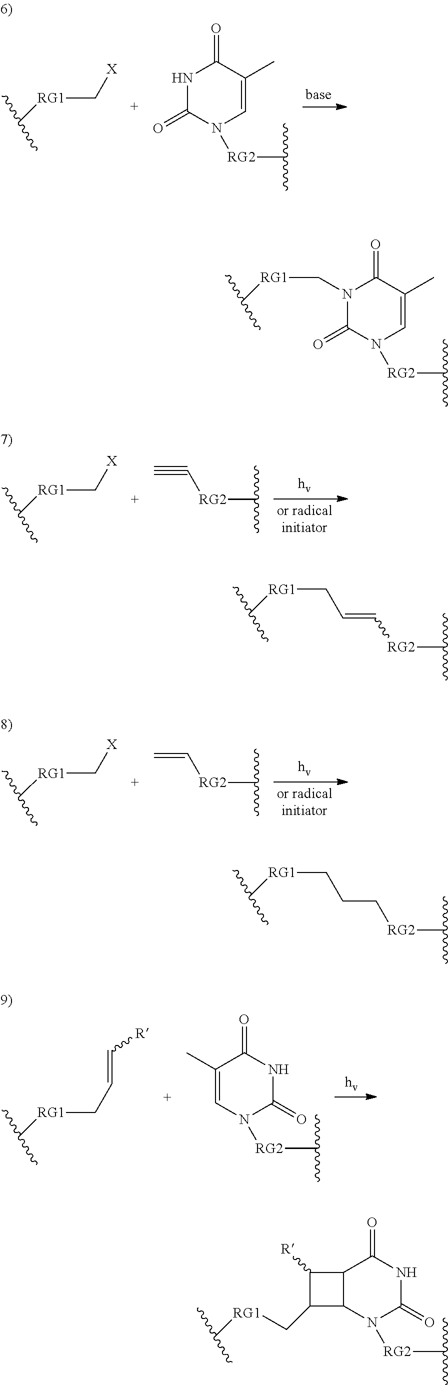

Referring back to Scheme 2, an example process for in situ probe inversion can be accomplished when using a branched linker (shown in FIG. 2A, or Alk), a cleavable linker (shown in FIG. 3C, or UCL), and bromohexyl phosphoramidite (shown in FIG. 4, or BromohexylLinker) as non-nucleotide reagents in a DNA synthesizer after employing oligothymidine groups in the surface treatment step of the substrate. Many synthetic steps with phosphoramidite reagents can be accomplished by the DNA synthesizer with or without modification of synthetic procedures of the machine for oligonucleotide synthesis. The DNA synthesis can be conducted using photolithography methods by employing photo-labile protecting groups on the reagents used for DNA synthesis.

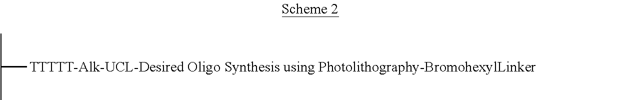

Oligothymidine linker (containing, for example, five (5) thymidines) can be added to the chip surface and further treatments can provide free hydroxyl groups at or near the distal end of the thymidine tail relative to the substrate surface. Incorporation of branched linker (shown in FIG. 2A) phosphoramidite to the free hydroxyl groups can be employed as a reagent in the DNA synthesis process to attach the branched linker to the surface of the substrate. After removing the DMT protecting group on the branched linker, the branched linker on the surface can comprise one branch of free hydroxyl group (“OH”) and another branch of alkyne.

Then cleavable linker (shown in FIG. 3C, also called universal cleavable linker) phosphoramidite can react with the free hydroxyl groups on the branched linker to afford covalently linked cleavable linker-branched linker complex. Removal of protecting group on the cleavable linker can provide a free hydroxyl group on the cleavable linker. These free hydroxyl groups on the cleavable linker thus obtained can serve as anchors for the standard DNA synthesis to provide a single stranded DNA probe sequence (or oligonuceotide), whose sequence can be pre-determined and/or tailored for the target nucleic acid molecules or other purposes of the array. Oligonucleotides can be introduced by any oligonucleotide synthesis method in a stepwise manner, for example, using the phosphoramidite chemistry and using photolithography method. Photolithographic in situ synthesis of DNA, using light projection from a digitally-controlled array of micromirrors, has been successful at both commercial and laboratory scales.

For each round of oligonucleotide synthesis, a capping step is performed to cap a free hydroxyl group after the oligonucleotide extension (since no oligonucleotide extension on this hydroxyl group, it can still be reactive) with an acetyl group or other suitable capping molecules. In this way capped failure oligo sequences can no longer be extended by the rest of chain elongation process for the full-length probe synthesis. Thus, these capped failure sequences can be truncated probes. As a result, some of the synthesized DNA probe sequences can be full length probe sequences while other can be truncated probe sequences at the end of the DNA synthesis. The last nucleotide coupling step for the full-length probe sequences can involve a non-nucleotide phosphoramidite—bromohexyl phosphoramidite (shown in FIG. 4, can also be referred to as “Br-Hex”) to provide a primary bromide at the 5′ end of the full-length probe sequences. None of the truncated probe sequences can have such a primary bromide tail on their 5′ end because they lack free hydroxyl groups for the ultimate reaction with the bromohexyl phosphoramidite reagent due to capping during prior DNA synthesis.

In situ probe inversion can occur when synthesized probes, both full-length probes and truncated probed, are treated with a base reagent, such as, for example, NH₄OH, ethylenediamine/water (EDA: water), or AMA (a mixture of NH₄OH and methyl amine). The base reagent may carry out multiple different reactions: circularization of full-length probe sequences to the substrate, cleavage of cleavable linkers for both full-length probe sequences and truncated probe sequences, and global deprotection of cyanoethyl protecting group on the phosphotriesters of the probe sequences and the remaining linkers. As a result, a free 3′-OH on the 3′ end of the full-length probe sequence with 5′ to 3′ orientation on the substrate can be obtained.

While knowing the exact mechanism of in situ probe inversion is not required to perform the method of the invention or limiting the present disclosure in any way, several mechanisms for in situ probe inversion may be possible: The first possible mechanism of in situ inversion can be that the base treatment of a thymidine group of the oligothymidine linker on the chip surface may deprotonate the hydrogen attached to the ring —NH-group (N(3)), which may attack the bromohexyl group at the 5′ end of full-length probe sequences in the presence of base, such as, for example, EDA:water or AMA, thereby making a covalent bond between the ring nitrogen (N(3)) of thymidine at the 3′ end of the full-length probe sequences and the 5′end thereof, followed by in situ cleavage of universal cleavable linker (UCL) to release free hydroxyl groups at the 3′ termini of the inverted full-length probe sequences. The second possible mechanism of in situ inversion can be that the base treatment of the branched linker may deprotonate the amide nitrogen of the branched linker to attack the bromohexyl group at the 5′ end of full-length probe sequences in the presence of base, such as, for example, EDA:water or AMA, thereby completing the inversion process discussed above. The third possible mechanism of in situ inversion can be that the base treatment of terminal acetylene may afford acetylide ion on the second branch of the branched linker to attack the bromohexyl group at the 5′ end of full-length probe sequences in the presence of base, such as, for example, EDA:water or AMA, thereby completing the inversion process discussed above. The fourth mechanism of in situ inversion can be photo-crosslinking between surface bound oligothymidine groups or other chemical groups on the surface and one or more oligo bases on the synthesized probe sequence. Other mechanisms can be possible. For example, photo-crosslinking of the probe sequence with oligothymidine tails coupled to the surface.

It should be noted that that the circularization can occur between a bromohexyl group and an acetylene group on the same branched linker, circularization can also happen between a bromohexyl group on one branched linker and an acetylene group on another branched linker. Likewise, the circularization reaction between a bromohexyl group and a thymidine group can happen on the same branched linker or between different branched linkers. Similarly the circularization reaction between a bromohexyl group and an amide nitrogen of a branched linker can happen on the same branched linker or between different branched linkers.

In one example, controlled pore glass (CPG) beads can be used as the synthesis substrate, which reacts with branched linker and cleavable linker. Then oligonucleotide probes can be synthesized on cleavable linkers attached to the substrate, including a terminal bromide on a linker at the 5′ end of the full-length probe sequence.

Linkers used, including branched linkers and cleavable linkers, can be stable during oligonucleotide probe synthesis processes such as those discussed herein. Stability of linkers can allow oligonucleotide probe synthesis without the use of protection-deprotection steps to preserve the linkers. Linkers can lack reactive species. The absence of reactive species from the linkers can result in the linkers being inert during a DNA synthesis process, which can remove the need for protection-deprotection steps. In some cases, at least about 10%, 20%, 30%, 40%, 50%, 60%, 70%, 75%, 80%, 85%, 90%, 95%, 96%, 97%, 98%, 99%, 99.9%, 99.99%, or 99.999% of linkers present prior to DNA synthesis remain intact after DNA synthesis.

The probe inversion techniques discussed herein can be conducted in aqueous media. Avoidance of the use of organic solvents can make such techniques more environmentally friendly and increase the ease of chemical handling and waste disposal.

The probe inversion techniques discussed herein can be conducted at a pH of at least about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, or 13.5. The probe inversion techniques discussed herein can be conducted at a pH of at most about 14.0, 13.5, 13.0, 12.5, 12.0, 11.5, 11.0, 10.5, 10.0, 9.5, 9.0, 8.5, 8.0, 7.5, 7.0, 6.5, 6.0, 5.5, 5.0, 4.5, 4.0, 3.5, 3.0, 2.5, 2.0, 1.5, 1.0, or 0.5. The probe inversion techniques discussed herein can be conducted at a pH of about 0.5, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, or 13.5. In some cases, the probe inversion techniques discussed herein can be conducted at or about physiological pH, such as about 7.365 or about 7.5. Conducting reactions at physiological pH can reduce or obviate the need for handling harsh substances or reaction conditions, and can employ aqueous media.

The probe inversion techniques discussed herein can be conducted at a temperature of about 15° C., 20° C., 25° C., 30° C., or 35° C. The probe inversion techniques discussed herein can be conducted at a temperature of at most about 15° C., 20° C., 25° C., 30° C., or 35° C. The probe inversion techniques discussed herein can be conducted at a temperature of at least about 15° C., 20° C., 25° C., 30° C., or 35° C. In some cases, the probe inversion techniques discussed herein can be conducted at or about room temperature, such as about 20° C., about 21° C., about 22° C., about 23° C., about 24° C., about 25° C., about 26° C., from about 20° C. to about 26° C., or from about 20° C. to about 22° C. Conducting reactions at room temperature can reduce or obviate the need for handling harsh substances or reaction conditions.

Releasing truncated probe sequences can increase the percentage of full-length sequences present in the array. In some cases, at least about 10%, 20%, 30%, 40%, 50%, 60%, 70%, 80%, 90%, 95%, 96%, 97%, 98%, 99%, 99.9%, 99.99%, or 99.999% of probes remaining bound to the array substrate following a probe inversion process are full-length sequences. In some cases, a probe inversion process can release at least about 10%, 20%, 30%, 40%, 50%, 60%, 70%, 80%, 90%, 95%, 96%, 97%, 98%, 99%, 99.9%, 99.99%, or 99.999% of truncated probes bound to the array substrate prior to the probe inversion process.

The synthesis substrate can comprise different forms or shapes, such as a bead or a flat array. The synthesis substrate can comprise any suitable material, including but not limited to glass (e.g., controlled pore glass), silicon, or plastic. Substrates can comprise polymer coatings or gels, such as a polyacrylamide gel or a PDMS gel. Gels and coatings can additionally comprise components to modify their physicochemical properties, for example, hydrophobicity. For example, a polyacrylamide gel or coating can comprise modified acrylamide monomers in its polymer structure such as ethoxylated acrylamide monomers, phosphorylcholine acrylamide monomers, betaine acrylamide monomers, and combinations thereof.

Inverted probes can provide many advantages over standard non-inverted probes, for a variety of applications. For example, as discussed above, probe inversion can remove most or all of undesired truncated probe sequences, thereby providing a population of inverted probes containing up to 100% full-length probe sequences. Additionally, inverted probes can have the 3′ OH group free, which can be beneficial for conducting enzymatic reactions (e.g., single or multiple base extension, ligase reaction, etc.). The inverted probes can also be used for sequencing by synthesis (SBS) process, among other applications.

### EXAMPLES

### Example 1—Probe Synthesis on Controlled Pore Glass

Branched linker phosphoramidites (see, e.g., FIG. 2A) having an alkyne moiety can be coupled to a dT-derivatized controlled pore glass (CPG) solid support on a DNA synthesizer using standard DNA synthesis protocols (see, e.g., FIG. 1). Then, universal cleavable linkers (UCL, see FIGS. 3A, 3B and 3C) can be attached to the branched linkers using standard procedures, followed by synthesis of a DNA sequence (e.g., an 18-mer or a 20-mer) on the DNA synthesizer. In the last oligonucleotide coupling step, 5′-bromohexyl phosphoramidite (Br-Hex, see FIG. 4) can be incorporated as a reactive group (see FIG. 1). DNA synthesis can be conducted using an ABI 394 DNA synthesizer using standard DNA synthesis protocols. Extended coupling time (3 minutes) can be used for all linker phosphoramidites (BL, UCL, and Br-Hex). Appropriate steps can be added according to needs in protecting group manipulations.

### Example 2—Probe Synthesis on Chip

DNA synthesis on chips (e.g., silanated glass surfaces) can be conducted similarly to the protocols described in G. H. McGall, F. A. Fidanza, Photolithographic Synthesis of Arrays, in Methods in Molecular Biology: DNA Arrays, methods and Protocols; J. B. Rampal, Ed.; Humana Press: Torowa, N.J., 2001; Vol. 170, 71-101. In order to make specific DNA features on the chips, photo-cleavable phosphoramidites can be used instead of standard DMT-protected phosphoramidites. The same 18-mer or 20 mer DNA sequence in Example 1 can be used in Example 2.

### Example 3—On-Wafer Probe Inversion

Oligonucleotide arrays can be synthesized on-wafer and can be inverted using the reactions as discussed herein. Briefly, a wafer scale synthesized invertible chip can be obtained as follows. The term “invertible chip” as used herein can refer to a chip which comprises all the required inversion linkers (e.g., branched linker (BL), universal cleavable linker (UCL) and bromohexyl linker (Br-Hex)) explained in the present disclosure. The chip can also be derivatized by oligothymidine (e.g., TTTTT) before conducting the reaction using the branch linker (BL). The probe sequences at different locations on the chip can be the same or can be different. For example, the probe sequences can be 20-mer DNA sequences (e.g., 5′-TAC GAT TCA GCC GAT ACA GC-3′, which is complementary to part of AM1 extension sequence in Example 4). After the last nucleotide of the probe sequence has been coupled to the extending probe sequences and the ensuing capping reaction is completed, 5′-bromohexyl phosphoramidite (Br-Hex, see FIG. 4) can be incorporated. Then the chip can be treated with EDA:H₂O (1:1, v/v) or EDA:EtOH (1:1, v/v) and incubated overnight at room temperature (e.g., 25° C.) to provide the inverted probe chip. The inverted probe can comprise a free OH at the 3′ end of the probe sequence.

One control chip can be an invertible chip that has undergone the whole probe synthesis, including the incorporation of the bromohexyl chain, but has not undergone the deprotection reaction (e.g., not reacting with EDA:H₂O or EDA: EtOH overnight). This control chip can comprise non-inverted probe sequence which does not have a free OH at the 3′ end of the probe sequence.

A negative control chip can be a non-invertible chip that does not comprise any inversion linkers described for the invertible chip, that has undergone the whole probe synthesis, including the incorporation of the bromohexyl chain, and that has undergone the deprotection reaction (e.g., reacting with EDA:H₂O or EDA: EtOH overnight). This negative control chip can comprise non-inverted probe sequence which does not have a free OH at the 3′ end of the probe sequence. The control chip and the negative control chip can be used in template-based extension (or primer extension) reactions described in Example 4.

### Example 4—On-Wafer Template-Directed Extension

The inverted probe chip, the control chip and the negative control chip obtained in Example 3 can be used in hybridization/primer extension reactions. All three chips (i.e., the inverted probe chip, the control chip and the negative control chip) can be hybridized with a primer comprising a sequence complementary to the inverted probe. For example, the primer can be an AM1 extension sequence, 3′-ddC/ATG CTA AGT CGG CTA TGT CGT TAC TAA TGA TGG AGA GAA G-5′ (ddC is 2′-3′-dideoxycytidine). The 3′ OH group of the AM1 extension sequence is blocked with a ddC group in order to avoid extension reactions on the primer in the presence of polymerase. The first 20-mer connected to ddC in the AM1 extension sequence is complementary to the 20-mer DNA sequences in the probes obtained in Example 3.

The hybridization can be conducted in 4× saline-sodium citrate buffer (SSC) by incubating at 50° C. for 1 h. Then excess un-hybridized primers (e.g., AM1 extension sequence) can be washed out with 4× SSC and finally with 1× primer extension buffer. For template-directed extension (or primer extension) reaction 100 μL of the extension reaction mixture comprising 2 μM dNTPs (Cy3-dUTP, dATP, dCTP, dGTP), and 20 units of Taq polymerase can be added to the container containing each chip. Each chip can be incubated at 50° C. for 1 h. Then each chip can be washed with 4× SSC three or four times and finally washed by incubating at 50° C. for 30 min in 4× SSC.

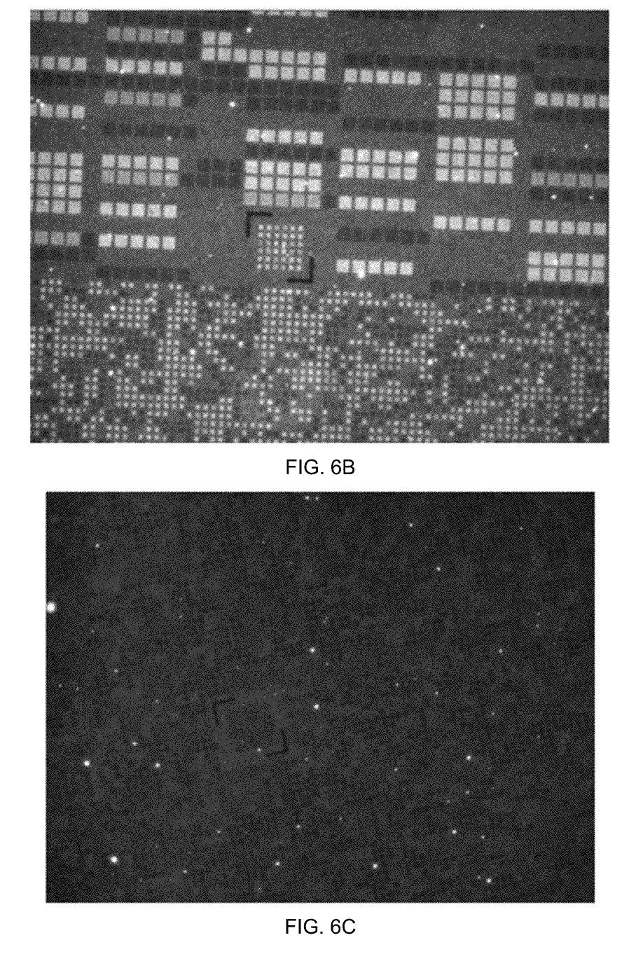

Results of the template-directed extension (or primer extension) reactions, or the lack thereof, can be visualized under fluorescence microscope, as shown in FIGS. 6A-6C (imaging at 40×, high resolution, 3 s exposure time). Fluorescence signals from extended oligonucleotides directed by the template AM1 extension sequence can be obtained from the invertible chip which has been proceeded through deprotection step, the inverted probe chip (see FIG. 6B). The invertible chip which does not go through the deprotection step, the control chip, cannot show any signals of template-directed extension (or primer extension) (FIG. 6A). Negative control chip cannot show any signals, either (FIG. 6C). Thus, FIG. 6B can show that the probe sequences introduced by DNA synthesizer can be inverted on the chip surface using the in-situ probe inversion method of the present disclosure, and that the inverted probe sequences can comprise free OH groups on their 3′ end to enable oligonucleotide synthesis when the probes are hybridized with primers/templates. FIG. 6A can show that without the deprotection step, no free 3′ OH groups are available on the control chip. FIG. 6C can show that without the invertible chip, no free 3′ OH groups are available even after conducting the deprotection step on the non-invertible chip.

### Example 5—In-Situ Probe Inversion and Testing

The chips comprising the synthesized oligonucleotide with our without the appropriate linkers were made similarly according to procedures described above.

1) Deprotection and Cleavage

The chips were incubated with 2 ml of EDA:H₂O (1:1) in a sealed box at room temperature overnight. Thereafter the chips were washed with water and acetonitrile.

2) Hybridization Experiment

The chips were hybridized with 0.5 μM 3′-blocked complementary DNA sequence (with respect to the synthesized oligonucleotide on the chips), having a few extra bases at the 5′-end (as template for the primer extension experiment in 3) below) and may comprising a fluorescent label at one of the ends, by incubating in 4× SSC at 50° C. for 1 h. The chips were then visualized to confirm hybridization.

3) Primer Extension Experiment

The chips obtained in 2) with the 3′-blocked complementary DNA sequence hybridized were washed thoroughly with 4×SSC and then with 1× Taq extension buffer. The chips were then incubated with extension reaction mixture containing 2 μM dNTPs (Cy3-dUTP, dATP, dCTP, and dGTP) and Taq polymerase (8U/μl) at 50° C. for 1 h. The chips were then washed several times with 4× SSC, and the final wash was done at 50° C. in 4× SSC for 30 min, followed by washing with water several times. The chips were then visualized to confirm chain elongation (primer extension on the 3′-end of the inverted probes).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of inverting an oligonucleotide on a surface, comprising:
(a) providing a substrate;
(b) coupling a branched linker to said substrate, wherein said branched linker comprises (i) a first branch comprising an acetylene moiety, and (ii) a second branch comprising a hydroxyl group;
(c) coupling a universal cleavable linker to said second branch via said hydroxyl group on said second branch;
(d) synthesizing a first oligonucleotide on said universal cleavable linker in 3′ to 5′ orientation, said first oligonucleotide comprising (i) a 3′ end coupled to said second branch via said universal cleavable linker, and (ii) a 5′ end coupled to a 6-bromohexyl linker;
(e) in-situ circularizing said first oligonucleotide by treating with a deprotection reagent, thereby coupling said 5′ end of said first oligonucleotide to said substrate; and
(f) in-situ cleaving said universal cleavable linker by said treating with said deprotection reagent, thereby de-coupling said 3′ end of said first oligonucleotide from said second branch.

2. The method of claim 1, wherein said deprotection reagent comprises a base.

3. The method of claim 2, wherein said base comprises an amine.

4. The method of claim 2, wherein said base comprises at least one of: (i) 1,2-diaminoethane, (ii) NH₄OH, and (iii) methyl amine.

5. The method of claim 1, further comprising in (d): building a second oligonucleotide in 3′ to 5′ orientation on a third branch on said substrate, wherein said second oligonucleotide is shorter than said first oligonucleotide and without another 6-bromohexyl linker coupled to a 5′ end of said second oligonucleotide, and wherein said in-situ cleaving in (f) releases said second oligonucleotide from said substrate.

6. A method of preparing probes on a substrate, comprising:
(a) coupling a plurality of first linkers to said substrate, wherein each of said plurality of first linkers is further coupled to a first branch comprising a first cleavable linker coupled to a 3′ end of a first oligonucleotide, and wherein a 5′ end of said first oligonucleotide is further coupled to a first reactive group;
(b) coupling a plurality of second linkers to said substrate, wherein each of said plurality of second linkers is further coupled to a second branch comprising a second reactive group; and
(c) circularizing said first oligonucleotide by reacting said first reactive group with said second reactive group, thereby coupling said 5′ end of said first oligonucleotide to said substrate via said second linker;
wherein said circularizing in (c) is conducted using nucleophilic substitution reaction, photo-crosslinking reaction, or radical reaction.

7. The method of claim 6, wherein said circularizing in (c) is conducted using nucleophilic substitution reaction.

8. The method of claim 7, wherein said circularizing is conducted using a base.

9. The method of claim 8, wherein said base comprises an amine.

10. The method of claim 8, wherein said base comprises at least one of: (i) 1,2-diaminoethane, (ii) NH₄OH, and (iii) methyl amine.

11. The method of claim 6, wherein said first reactive group comprises at least one of: (i) bromide, (ii) iodide, (iii) mesylate, (iv) tosylate, and (v) triflate.

12. The method of claim 6, wherein said second reactive group comprises at least one of: (i) terminal acetylene, (ii) thymidine, (iii) —C(O)NH—, (iv) amine, (v) —OH, (vi) alkene, and (vii) alpha-hydrogen of a carbonyl.

13. The method of claim 6, wherein said first reactive group is bromide.

14. The method of claim 6, further comprising: after (c), cleaving said first cleavable linker, thereby de-coupling said 3′ end of said first oligonucleotide from said first linker.

15. The method of claim 14, wherein both said circularizing and said cleaving are conducted using a base.

16. The method of claim 14, further comprising: before (c), coupling a plurality of third linkers to said substrate, wherein each said third linker is further coupled to a third branch comprising a second cleavable linker coupled to a 3′ end of a second oligonucleotide, wherein said second oligonucleotide is shorter than said first oligonucleotide, and wherein said cleaving releases said second oligonucleotide from said substrate.

17. The method of claim 16, wherein said second oligonucleotide does not comprise said first reactive group at a 5′ end of said second oligonucleotide.

18. A method of preparing probes on a substrate, comprising:
(a) coupling a plurality of first linkers to said substrate, wherein each said first linker is further coupled to a first branch comprising a first cleavable linker coupled to a 3′ end of a first oligonucleotide, and wherein a 5′ end of said first oligonucleotide is further coupled to a first reactive group;
(b) coupling a plurality of second linkers to said substrate, wherein each said second linker is further coupled to a second branch comprising a second reactive group; and
(c) circularizing said first oligonucleotide by reacting said first reactive group with said second reactive group, thereby coupling said 5′ end of said first oligonucleotide to said substrate via said second linker;
wherein said circularizing in (c) is conducted using a base.

19. The method of claim 18, wherein said base comprises an amine.

20. The method of claim 18, wherein said base comprises at least one of: (i) 1,2-diaminoethane, (ii) NH₄OH, and (iii) methyl amine.


SEQUENCE LISTING (2 sequences)

SEQ ID NO: 1 - DNA, 20 bp, Artificial Sequence
  Description of Artificial Sequence Synthetic probe
tacgattcagccgatacagc

SEQ ID NO: 2 - DNA, 41 bp, Artificial Sequence
  Description of Artificial Sequence Synthetic primer
  modified_base (1)..(1): 2′-3′-dideoxycytidine
catgctaagtcggctatgtcgttactaatgatggagagaag